(19) United States
(12) Patent Application Publication
(10) Pub. No.: US 2015/0064263 A1
(43) Pub. Date: Mar. 5, 2015

(54) COMPOSITIONS AND METHODS FOR REDUCTION OF MERCURY TOXICITY

(71) Applicant: David Kossor, Kingsport, TN (US)

(72) Inventor: David Kossor, Kingsport, TN (US)

(21) Appl. No.: 14/371,332

(22) PCT Filed: May 25, 2011
(86) PCT No.: PCT/US11/37957

§ 371 Date: Sep. 24, 2014

Related U.S. Application Data
(60) Provisional application No. 61/347,819, filed on May 25, 2010.

(51) Int. Cl. A61K 31/4045; A61K 9/50; A61K 35/74; A61K 36/06; A61K 33/30; A61K 31/555

(52) U.S. Cl. CPC A61K 31/4045; A61K 33/30; A61K 31/555; A61K 35/741; A61K 36/06; A61K 9/5005
USPC 424/497; 424/641; 424/490; 424/93.4; 424/93.51; 424/498

(57) ABSTRACT

Provided are compositions containing melatonin and zinc. Additionally provided are dosage forms containing the compositions, and methods of making the compositions and dosage forms. Methods of removing mercury from the body of subject are provided, as are methods of treating and/or preventing certain conditions associated with mercury toxicity.

### CROSS-REFERENCE TO RELATED APPLICATIONS

This application claims the benefit of U.S. Provisional Application No. 61/347,819, filed May 25, 2010, the disclosure of which is hereby incorporated by reference herein in its entirety.

### BACKGROUND OF THE INVENTION

Autism Spectrum Disorders (“ASD”) are a spectrum of psychological conditions characterized by widespread abnormalities of social interactions and communication, as well as restricted interests and repetitive behavior. The five forms of ASD include classic autism, Asperger syndrome, Pervasive Developmental Disorder Not Otherwise Specified (“PDD-NOS”), Rett syndrome, and childhood disintegrative disorder. Autism forms the core of the autism spectrum disorders. Asperger syndrome is closest to autism in signs and likely causes; however, unlike autism, people with Asperger syndrome have no significant delay in language development. PDD-NOS is diagnosed when the criteria are not met for a more specific disorder. Some sources also include Rett syndrome and childhood disintegrative disorder, which share several signs with autism but may have unrelated causes; other sources combine ASD with these two conditions into the pervasive developmental disorders. According to the National Autistic Society of the United Kingdom, Pathological Demand Avoidance syndrome belongs and is increasingly being recognized as belonging to the autistic spectrum.

Autism is a disorder of neural development characterized by impaired social interaction and communication, and by restricted and repetitive behavior. These signs all begin before a child is three years old. Autism affects information processing in the brain by altering how nerve cells and their synapses connect and organize; how this occurs is not well understood.

Melatonin is a naturally occurring compound found in animals, plants, and microbes. In animals, circulating levels of the hormone melatonin vary in a daily cycle, thereby allowing the entrainment of the circadian rhythms of several biological functions. Many biological effects of melatonin are produced through activation of melatonin receptors, while others are due to its role as a pervasive and powerful antioxidant, with a particular role in the protection of nuclear and mitochondrial DNA.

In mammals, melatonin is secreted into the blood by the pineal gland in the brain. It may also be produced by a variety of peripheral cells such as bone marrow cells, lymphocytes and epithelial cells. Usually, the melatonin concentration in these cells is much higher than that found in the blood but it does not seem to be regulated by the photoperiod.

Additionally, melatonin is produced throughout the gastrointestinal (GI) tract. Unlike in the pineal gland, where its production and release is stimulated by a decrease in ambient light (i.e., the “sleep” signal), GI melatonin release is stimulated by the presence of food. There are two principal mechanisms by which melatonin communicates with cells. One is directly, via its interaction with membrane and nuclear receptors. Additionally, data indicates that melatonin is an endogenous agonist for the so-called nuclear “retinoid orphan receceptor” (ROR). Stimulated by melatonin, the ROR is a central component by which many biochemical processes are regulated. From the ROR, a plethora of genes are activated/regulated, and as a result, hormones, cytokines, neurotransmitters and biologically-relevant macromolecules are produced and released in a coordinated, cyclical (circadian) fashion throughout the day/night. In short, the role played by pineal melatonin from the brain is a part of a much larger system that is centered in the GI system.

Mercury (represented by the symbol Hg) is a toxic heavy metal. The federal Agency for Toxic Substances and Disease Registry (ATSDR), within the Department of Health and Human Services, lists mercury as the most toxic substance in the United States. It is not a rare or isolated compound; in terms of sheer quantities, it is ranked as the third most prevalent environmental toxicant, after lead and arsenic. The dangers it poses are multiplied, many times over, by the fact that nearly any and all types of fish, poultry, and livestock which ingest low levels of mercury, in their own food sources, will concentrate the mercury in their flesh and/or fatty deposits. This leads to potentially dangerous levels of mercury in numerous types of meat that are major components of human diets.

Briefly, mercury kills cells mainly through two distinct mechanisms:

(i) it can “denature” (and thereby inactivate) nearly all types of proteins; and,

(ii) it creates “reactive oxygen species” (ROSs), which then attack and damage or destroy numerous types of biomolecules.

In addition, studies have linked mercury toxicity to certain conditions, including, but not limited to, Autism Spectrum Disorders.

### SUMMARY OF THE INVENTION

In certain aspects, the present invention relates to relates to compositions comprising melatonin and zinc.

In further aspects, the invention relates to dosage forms comprising the compositions.

Further aspects of the invention relate to methods of removing mercury from the body of a subject.

Additional aspects of the invention relate to methods of treating and/or preventing conditions associated with mercury toxicity.

Additional aspects of the invention relate to methods of making compositions and dosage forms described herein.

### DETAILED DESCRIPTION OF THE INVENTION

Certain embodiments of the invention relate to compositions and methods useful for removing mercury from the body of a subject. In certain embodiments of the invention, a nutritional approach to the treatment and prevention of conditions associated with mercury toxicity is provided.

Studies have shown that in the GI tract, there is an equilibrium that is created and maintained, wherein melatonin produced by enterochromaffin cells of the gut enter the splanchnic circulation and are transported to the liver; in turn, the liver secretes the melatonin into the bile which is transported back to the intestines to repeat the cycle. This so-called “enterohepatic cycle” is the basis for the principal indirect hormonal action of melatonin in the GI tract. Within this enterohepatic cycle, melatonin behaves as a “metallochaperone” as it complexes with trace metals (principally zinc and copper) to facilitate their absorption from the GI tract and their delivery to the liver. Once in the liver, the melatonin-metal complex dissociates, to release the metals in the vicinity of apoproteins, to ultimately produce fully-functional metalloproteins. Zinc metalloproteins include a number of important macromolecules; for example, matrix metalloprotinases, DNA/RNA polymerases and repair enzymes, carbonic anhydrases, amino/carboxyltranspeptidases, and alcohol dehydrogenase.

As a divalent cation, mercury effectively competes with zinc (and copper) for binding with melatonin, and when mercury binds to melatonin, the relatively heavier complex is not reabsorbed and transported to the liver, but rather remains in the fecal contents of the gut, to be eliminated. By protecting the GI tract from the harmful effects of mercury (and other heavy metals), the melatonin is lost from the body, which must manufacture more melatonin to maintain endogenous concentrations. While not intending to be bound by any theory of operation, one or more subsets of the population, for a variety of reasons, may lack the capacity to regenerate adequate amounts of melatonin to maintain the balance, and thus, represent those who may be predisposed to develop diseases (including, but not limited to, autism, Alzheimer's disease, and Parkinson's disease) that are due, in part, to exposure to environmental heavy metals. Such subgroup(s) also represent those who may benefit from the administration of compositions comprising melatonin.

Ingesting melatonin-containing compositions described herein may (a) provide a treated subject with an effective mercury-chelating and mercury-removal compound, and/or (b) provide a treated subject with a sufficient supply of gastrointestinal melatonin to restore the proper and healthy functions and activities of gastrointestinal melatonin, which can interact with zinc and certain other minerals. As such, certain embodiments of the invention relate to compositions and methods for treatment and/or prevention of certain conditions using melatonin. Such compositions and methods may restore natural and healthy concentrations of melatonin within the gastrointestinal tract.

In certain embodiments, melatonin may be used conjunction with certain additional ingredients. Additional ingredients include, but are not limited to, zinc and copper. Various embodiments may include the combination of melatonin with minerals in particular ratios.

Certain embodiments provide compositions comprising a melatonin-zinc complex. Additionally, in certain embodiments, the compositions may further comprise probiotic materials.

Accordingly, one embodiment of the invention includes a composition comprising melatonin and zinc. Preferably, the zinc may be provided as at least one pharmaceutically acceptable salt thereof. In preferred embodiments, the form of zinc may be selected from a group consisting of zinc, zinc acetate, zinc gluconate, zinc citrate, zinc chloride, and combinations thereof. In a preferred embodiment, the melatonin and zinc are mixed in a molar ratio from about 1:1 to about 1:1.2 (i.e., a potential molar excess of divalent zinc). Preferably, the melatonin and zinc are present in about a 1:1 molar ratio.

In a preferred embodiment, the melatonin and zinc are in a powdered form and mixed into a powdered mixture. The powdered mixture may be formulated with an amount of at least one excipient sufficient to produce particles. Common excipients that may be used for this purpose include, but are not limited to, carbohydrate materials. Preferably, the carbohydrate material is in powdered form. One example of a carbohydrate that may be used for this purpose is lactose, also known as milk sugar. Additional examples include, but are not limited to, cellulose derivatives and potato starch. Care should be taken to minimize the presence of myoinositol hexaphosphate, also known as phytic acid (or as its ionized form, phytate). This compound is known to form complexes with zinc and other divalent cations, and may thus interfere with its bioavailability. For this reason, certain materials may be less suitable for this purpose, such as, for example, corn starch, since myoinositol hexaphosphate is known to be produced by corn and may be present in corn starch as a contaminant.

Preferably, the particles may be coated with an enteric coating. In certain embodiments, the enteric coated particles may be encapsulated. Preferably, a gelatin capsule may be used for encapsulation.

In certain embodiments, the zinc and melatonin are pre-mixed with each other, to allow them to become efficiently bound to each other. In a preferred embodiment, the mixture is coated with an enteric coating that will pass unharmed through the stomach acid and then be digested within the small intestine; this may protect the melatonin against degradation by stomach acids, and it also may minimize acid-driven dissociation of zinc ions from the melatonin in the stomach.

An “enteric coating” refers to a barrier applied to oral medication that controls the location in the digestive system where it is absorbed. “Enteric” refers to the small intestine, therefore enteric coatings prevent release of medication before it reaches the small intestine.

Most enteric coatings work by presenting a surface that is stable at the highly acidic pH found in the stomach, but breaks down rapidly at a less acidic (relatively more basic) pH. For example, they will not dissolve in the acidic juices of the stomach (pH˜3), but they will in the alkaline (approximately pH 5-9) environment present in the small intestine. With regard to embodiments of this invention, the materials useful for enteric coatings may comprise any suitable coating material that will preferentially dissolve in the intestines (i.e., at a pH that is greater than that of the contents of the stomach). Such materials include, but are not limited to, fatty acids, waxes, shellacs, plastics, plant fibers, polymers, and combinations thereof. A preferred material is a dispersion of pH-sensitive acrylic polymer resin; for example, EASTACRYL 30D® (Eastman Chemical Co.).

In certain embodiments, the composition may further comprise a therapeutically effective amount of copper.

Accordingly, another embodiment of the invention relates to a composition comprising melatonin, zinc and copper. In various embodiments, the melatonin, zinc and copper components are in a molar ratio of about 5:5:1; about 6:6:1, about 7:7:1, about 8:8:1, about 9:9:1, or about 10:10:1, respectively. Preferably, the melatonin, zinc and copper components are in a molar ratio of about 5:5:1, respectively.

In another preferred embodiment, the melatonin, zinc and copper components are in a powdered form and mixed into a powdered mixture. The powdered mixture may be formulated with a sufficient amounts of at least one excipient to produce particles. Preferably, the particles may be coated with an enteric coating. Preferably, the enteric coating comprises EASTACRYL®. Preferably, the enteric coating my further comprise additional coating materials that dissolve in the upper portion of the small intestines.

In a preferred embodiment, the enteric coated particles are encapsulated, preferably in a gelatin capsule. A sufficient quantity of particles may be placed into each capsule to maintain the final molar ratios of melatonin, Zn, and Cu. In a preferred embodiment, based on their molecular weights, the corresponding amounts of melatonin, zinc acetate and Cu which deliver the proper molar ratios are about 50 mg, about 15 mg, and about 3 mg, respectively, when measured as elemental weights. In further embodiments, these weights can be adjusted to account for molecular weight differences among the various zinc salts that may be used in the composition.

When the compositions described herein are formulated with an enteric coating, the enteric coating protects ingredients in the composition from the harmful acid environment of the stomach. Subsequent to their transit through the stomach, the enteric coated particles enter the duodenum (the upper portion of the small intestine), where they encounter biliary secretions. The biliary secretions are rich in bicarbonate, which neutralizes the acid from the stomach contents as it emerges from the stomach, and causes the material in the lumen of the duodenum to become less acidic, with a pH greater than about 5. Thus, at this relatively higher pH, the enteric coating dissolves, enabling the dissolution of the particles, leading to the absorption of components such as melatonin, Zn, and Cu from the lumen of the small intestine.

In additional embodiments of the invention, melatonin and zinc provided in the composition may be in the form of a melatonin-zinc complex. An example of a stable zinc-containing complex suitable for oral administration is polaprezinc, described in U.S. Pat. No. 4,981,846. In a preferred embodiment, the melatonin-zinc complex may be formed by adding a soluble zinc salt to an alkaline solution of melatonin in the presence of excess alkali. While not intending to be bound by any theory of operation, such a complex may be formed as follows. The structure of melatonin has nitrogen and oxygen atoms that are electronegative due to lone pair electrons. In the melatonin structure, the pair of electrons on the oxygen are available to interact with other molecules (such as hydrogen bonding with water molecules) and form resonance structures via ionic interactions. However, divalent zinc has two positive charges, and to form a resonance-stabilized complex, it needs two sites of interaction with a macromolecule. For melatonin, the second site of interaction for zinc is provided by the electrons of a secondary nitrogen in the nearby heterocycle. Unlike oxygen, which stabilizes these electrons in a double bond, the nitrogen exists in a protonated form (i.e., with a hydrogen atom that shares an ionic bond with the nitrogen). To expose these electrons to provide a docking site for the zinc, the hydrogen needs to be removed from the nitrogen, and this is accomplished by dissolving the melatonin in an alkaline medium (i.e., an aqueous solution containing sodium hydroxide, calcium hydroxide, or other suitable alkaline material). In this environment, the alkali effectively removes the hydrogen from the nitrogen, thereby exposing the electrons that are available to interact with zinc that is added subsequently.

Additional embodiments include methods of removing mercury from the body of a subject.

In further embodiments, the invention relates to compositions and methods of treatment using probiotic nutritional supplements that contain or express certain types of microbial enzymes capable of converting damaging ionic forms of mercury (Hg⁺ and Hg²⁺) into uncharged elemental mercury atoms (Hg⁰), which can be excreted by the body in gaseous form, for example, in air from the lungs. In certain embodiments, compositions may comprise enzyme preparations having enteric coatings which protect the enzymes against degradation by stomach acids. In additional embodiments, compositions may comprise viable enteric bacterial cells which reproduce within the small intestines and express mercury-detoxifying enzymes.

A particular cluster of bacterial enzymes has been identified, which are produced by a cluster of genes called “the mer operon”. One enzyme which is encoded by that operon is “mercuric reductase” (“MR”). It catalyzes the reduction of the highly toxic divalent ion, Hg²⁺, to the uncharged and much less toxic Hg⁰. The MR enzyme is expressed by a gene designated as merA.

In addition, some types of bacteria also express an enzyme called organomercurial lyase (“OL”). That enzyme will cleave organic forms of mercury, such as methylmercury, to release the Hg²⁺ cation, which can then be converted into Hg⁰ by the mercuric reductase enzyme. OL is encoded and expressed by the merB gene.

Certain other genes within the mer operon encode translocase proteins, including proteins designated as merB and merC, which facilitate the movement of mercurial compounds across cell membranes, thereby facilitating their interactions with the OL and MR enzymes. Thus, proteins expressed by the merB and merC genes provide synergistic support for MR activity, by means of a multi-step process involving: (i) increased cellular uptake of mercurial compounds; (ii) cleavage of organic mercury compounds to release Hg²⁺ ions; and, (iii) reduction of the toxic Hg²⁺ ions into much less toxic Hg⁰ elemental form, which can be excreted by the body in gaseous form in air from the lungs.

Certain embodiments of the invention include compositions comprising melatonin, zinc, and probiotic materials. Probiotic materials may include, without limitation, natural, semisynthetic, or transgenic strains of bacteria and/or yeast. The probiotic materials may comprise or express certain mercury-detoxifying enzymes such as, without limitation, organomercurial lyase, and mercuric reductase enzyme.

In certain embodiments, compositions are provided comprising melatonin, zinc, copper, and probiotic materials. Additional embodiments provide compositions comprising a melatonin-zinc complex and probiotic materials.

In certain embodiments, the invention provides dosage forms comprising the compositions described herein. Solid dosage forms for oral administration may include, but are not limited to, capsules, tablets, pills, and granules.

Granules may be preferable for certain patients such as, for example, children, who might have difficulty swallowing larger dosage forms. To facilitate an exact dose, in a preferred embodiment the requisite number of granules are transferred to a standard gelatin capsule, and at the time of ingestion, the patient can elect to open the capsule and disperse the contents into a suitable food such as, for example, applesauce, which is sufficiently acidic to preserve the enteric coat of the granules immediately prior to ingesting. Alternatively, the standard capsule with its content of granules intact can be ingested as a single dose, assuming that the patient has no difficulty in swallowing the capsule.

In a preferred embodiment, the dosage form comprises a combination of active ingredients contained within a capsule. Preferably, the capsule comprises gelatin. The capsule may be hard or soft.

Further embodiments of the invention include methods of treating and/or preventing a condition associated with mercury toxicity. Such a condition may be associated with depletion of melatonin and/or zinc. Embodiments include the treatment and/or prevention in a subject of a condition selected from the group consisting of autism, autism spectrum disorders, Alzheimer's disease, and Parkinson's disease. Additional embodiments include methods of treatment and/or prevention of a condition selected from the group consisting of inflammation, mitochondrial dysfunction, and zinc deficiency. Another embodiment of the invention includes a method of treating and/or preventing a condition associated with exposure to environmental heavy metals.

The foregoing methods of treatment/prevention may comprise administering to a subject in need thereof a therapeutically effective amount of any of the compositions disclosed herein.

As used herein, the term “subject” is used to mean an animal; including, but not limited to, fish, avian and mammal, including a human. The terms “patient” and “subject” may be used interchangeably.

Additionally, “therapeutically effective amount” as used herein shall mean that dosage that provides the specific pharmacological response for which an agent or ingredient is administered in a significant number of subjects in need of such treatment. It is emphasized that the “therapeutically effective amount” administered to a particular subject in a particular instance will not always be effective in treating or preventing the conditions described herein, even though such dosage is deemed a “therapeutically effective amount” by those skilled in the art.

Techniques for formulation and administration of the therapeutic compositions of the instant application may be found in “Remington's Pharmaceutical Sciences,” Mack Publishing Co., Easton, Pa., latest edition. When applied to an individual active ingredient or agent, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

When treating or preventing any of the listed conditions or symptoms, the compositions described herein may preferably be administered orally. Preferably, the compositions may be administered one, two, three or four times a day. Preferably, the compositions may be orally ingested with meals, to mimic the physiological release of melatonin by the presence of food in the GI tract. A preferred dosage is four times per day; preferably, once with each meal and once at bedtime.

In various embodiments, the subject may be administered a dosage of a composition described herein an amount of at least about 0.01 mg calculated according to the amount of melatonin in the composition per kg weight of the subject per dose; for example, dosage ranges of composition that provide from about 0.01 mg/kg to about 10 mg/kg melatonin, or about 0.02 mg/kg to about 0.5 mg/kg melatonin, per dose. A preferred dose is one that provides about 0.1 mg/kg melatonin. One or more additional components, (e.g., zinc, copper) may be included in the composition as described herein according to the molar ratios provided. Preferably, the dosage may be administered one, two, three or four times per day. The exact dosing regimen may depend upon an individual patient's response, as determined, for example, by a health practitioner.

Further embodiments of the invention include methods of making the various compositions and dosage forms described herein. Preferably, a dosage form is prepared by a method comprising combining melatonin and zinc in powdered form to form a powdered mixture. The molar ratio may be from about 1:1 to about 1:1.2. Preferably, the melatonin and zinc are in about a 1:1 ratio. Preferably, said powdered mixture may be combined with at least one inert excipient in an amount sufficient to form particles. Preferably, the particles may be coated with a material that dissolves in media with a pH greater than or equal to about 5. In certain embodiments, the coated particles may be encapsulated in a capsule. Preferably, the capsule comprises gelatin.

The following non-limiting example(s) set forth herein below illustrate certain aspects of the invention.

### EXAMPLES

### Example 1

### Protocol to Assess the Efficacy of Melatonin and Zinc in ASD Patients

This example includes simulated tests and predicted results which can be conducted based on the description of this specification by those skilled in the art at the time of filing this application. Male and female children between the ages of 3 to 10 with autism or ASD with parental or teacher concerns with externalizing behaviors and with parental or clinician concerns with gastroenterologic problems are used in this prophetic example. Specifically, the children exhibit the following characteristics: (1) children from 3 to 10 years of age with diagnosed ASD; (2) parental or teacher concern with externalizing behaviors (with CBCL externalization t score>65); (3) parental or clinician concerns with gastroenterologic concerns (constipation, diarrhea, nausea, emesis, abdominal distress, low body mass index, food intolerance); (4) one week dietary diary suggests age-typical fiber, protein, fat, calorie and nutrient intake (no values are more than 2 Standard Deviation from average).

Efficacy of the melatonin and zinc compositions will be measured by observing behavior changes in the subjects using behavioral assessments from baseline to week 7/Termination, determined by standardized behavioral testing methodologies, including (1) the CBCL; (2) the Autism Treatment Evaluation Checklist (ATEC)(Lonsdale, D., Shamberger, R. J., and Audhya, T. (2002). Treatment of autism spectrum children with thiamine tetrahydrofurfuryl disulfide: a pilot study. Neuro. Endocrinol. Lett. 23, 303-308; Charman, T., Howlin, P., Berry, B., and Prince, E. (2004). Measuring developmental progress of children with autism spectrum disorder on school entry using parent report. Autism. 8, 89-100; Ratliff-Schaub, K., Carey, T., Reeves, G. D., and Rogers, M. A. (2005). Randomized controlled trial of transdermal secretin on behavior of children with autism. Autism. 9, 256-265; Coben, R., and Myers, T. E. (2010). The relative efficacy of connectivity guided and symptom based EEG biofeedback for autistic disorders. Appl. Psychophysiol. Biofeedback. 35, 13-23; Meiri, G., Bichovsky, Y., and Belmaker, R. H. (2009). Omega 3 fatty acid treatment in autism. J. Child Adolesc. Psychopharmacol. 19, 449-451); (3) the Clinical Global Impressions of Behavior-Severity (CGI-S) and (4) the Clinical Global Impressions of Behavior-Improvement (CGI-I). Secondary Outcome Measures include changes between baseline and week 7/termination values for (1) GI permeability that will be assessed by measuring changes in the urinary lactulose:mannitol ratios following a single oral dose of these carbohydrates (D'Eufemia, P., Celli, M., Finocchiaro, R., Pacifico, L., Viozzi, L., Zaccagnini, M., Cardi, E., and Giardini, O. (1996). Abnormal intestinal permeability in children with autism. Acta Paediatr. 85, 1076-1079); (2) GI inflammation that will be assessed by measuring fecal calprotectin levels (Boso, M., Emanuele, E., Minoretti, P., Arra, M., Politi, P., Ucelli di, N. S., and Barale, F. (2006). Alterations of circulating endogenous secretory RAGE and S100A9 levels indicating dysfunction of the AGE-RAGE axis in autism. Neurosci. Lett. 410, 169-173; deMagistris, M. L., Familiari, V., Pascotto, A., Sapone, A., Frolli, A., Iardino, P., Carteni, M., De, R. M., Francavilla, R., Riegler, G., Militerni, R., and Bravaccio, C. (2010). Alterations of the Intestinal Barrier in Patients With Autism Spectrum Disorders and in Their First-degree Relatives. J. Pediatr. Gastroenterol. Nutr.); (3) Feeding and Gastroenterologic Checklist total and subscale score changes; (4) changes in height percentiles; (5) changes in weight percentiles; (6) changes in body mass index percentiles; (7) clinical chemistry parameters, including concentrations of serum zinc, plasma copper, and plasma 5′-nucleotidase activity; genomic evaluations of specific single nucleotide polymorphisms (SNPs) in the ASMT gene.

The screening/baseline evaluations will include a thorough evaluation for neurodevelopmental/psychiatric disorders; an evaluation of physical disorders; medication and supplementary treatment history; confirmation of the diagnosis of autism (with the Autism Diagnostic Observation Schedule); behavioral measures; specific lab tests; evaluation of gastrointestinal problems; evaluation of diet; assessment of suicidality; measures of daytime sleepiness.

Once the child is determined to meet the necessary criteria, he or she will be assigned to either the treatment group (Group 1) or the placebo group (Group 2). Subjects in group 1 will receive a target dose of 0.10 mg melatonin+0.03 mg zinc acetate dihydrate/kg/dose qid, and subject in group 2 will receive placebo qid. The medication will be provided as enteric-coated granules that will be dispersed in food prior to oral administration.

For week 1, the child will be administered a single dose of study medication at bedtime daily for one week. Follow-up assessments for dose escalation will take place at 7 day intervals (±2 days) for at least three weeks; at each weekly visit, the child will have evaluation of interval behavioral, gastroenterologic, and medical changes; adverse events will be elicited, recorded, and managed; concomitant medications will be elicited and recorded; behavioral checklists will be completed; assessment of any suicidality tendencies; assessment of daytime sleepiness.

At each visit, the most appropriate dose adjustment will be determined according to the treatment effect:

(1) intolerable: CGI-I score of 6 or 7 related to study interventions, or the presence of an adverse event that does not have available study-permitted remediation (e.g., requires medications that are not permitted). The study medication dose is to be reduced or the study medication is to be discontinued, based on the clinical decision of the investigator.

(2) remediable: CGI-I score of 6 or 7 related to a transient or remediable concern, or an adverse event that is transient or has available study-permitted remediation. The study medication may be either reduced, discontinued, or maintained at the discretion of the investigator.

(3) inadequate: minimal or no significant change in either gastroenterologic or behavioral status (no clinically significant improvement in the Feeding and Gastroenterologic Checklist with CGI-I score of 3, 4, or 5) without clinically significant adverse events. The study medication will be increased to the next available dose without exceeding the maximum dose for the treatment group.

(4) adequate: clinically significant improvements in either gastroenterologic or behavioral status (clinically significant improvement in the Feeding and Gastroenterologic Checklist and/or CGI-I score of 2) without clinically significant adverse events. At the discretion of the investigator, the study medication may be maintained at the current dose or may be increased (to try to attain optimal status) to the next available dose without exceeding the maximum dose for the treatment group.

(5) optimal: clinically significant improvements in the gastroenterologic and behavioral status (clinically significant improvement in the Feeding and Gastroenterologic Checklist and CGI-I score of 1) without clinically significant adverse events. The study medication will be maintained.

When a child is assessed by the investigator to fit into classification (2) remediable, the titration process may be extended by an additional week to permit optimal titration. For instance, if the study medication is increased at Week 2 and the child shows mild daytime somnolence, the investigator may assess the treatment classification as (2) remediable and may continue the present dose for an additional week to determine if the daytime somnolence resolves as the child become “used” to the medication.

Expected dosing schedule. At Baseline, the child will be administered 0.1 mg/kg (group 1) or placebo (group 2) at bedtime for one week. Then, at the Week 1 visit, the child will be 0.1 mg/kg or placebo morning and at bedtime for one week. At the Week 2 visit, the child will be administered 0.1 mg/kg or placebo morning, lunchtime, and at bedtime for one week. At the Week 3 visit, the child will be administered 0.1 mg/kg or placebo morning, lunchtime, dinnertime, and at bedtime

TABLE 2
Schedule of Study Activities

| | | | | | | | Post- | Post- |
|---|---|---|---|---|---|---|---|---|
| | Baseline | Week 1 | Week 2 | Week 3 | Week 4 | Week 7 | term | study |
| VS | + | + | + | + | + | + | + | + |
| DEval | + | + | + | + | + | + | + | + |
| MEval | + | + | + | + | + | + | + | + |
| PriorP | + | | | | | | | |
| PriorG | + | | | | | | | |
| ADOS | + | | | | | | | |
| ABC | + | + | + | + | + | + | | |
| CGI-S | + | | | | + | + | | |
| CGI-I | | + | + | + | + | + | | |
| F-cal | + | | | | | + | | |
| M:L | + | | | | | + | | |
| Labs | + | | | | | + | | |
| SNPs | + | | | | | | | |
| Diary | + | | | | + | + | | |
| FGC | + | + | + | + | + | + | | |
| C-SSRS | + | + | + | + | + | + | | |
| CHQ | + | | | | + | + | | |
| ESS | + | + | + | + | + | + | | |
| MedDisp | + | + | + | + | + | + | | |
| MedColl | | + | + | + | + | + | + | |
| AE | | + | + | + | + | + | + | + |
| ConMed | | + | + | + | + | + | + | + |
| TE | | + | + | + | + | | | |

Vital signs, growth parameters, calculation of body mass index (VS)
Neurodevelopmental, Developmental-Behavioral, or Child Psychiatric evaluation (DEval)
Pediatric general medical evaluation with review of systems, medical history, and physical exam (MEval)
Review of prior psychologic and psychopharmacologic treatments (PriorP)
Review of prior gastroenterologic treatments (PriorG)
Autism Diagnostic Observation Schedule (ADOS)
Aberrant Behavior Checklist (ABC)
Clinical Global Impressions of Behavior - Severity (CGI-S)
Clinical Global Impressions of Behavior - Improvement (CGI-I)
Fecal calprotectin levels (F-cal, two times one week apart)
Urinary mannitol/lactulose ratio (M:L)
Labs (serum [Zn], plasma [Cu], plasma 5′ nucleotidase activity, serum lipid peroxidation).
Analysis of single nucleotide polymorphisms (SNPs) in genes related to melatonin biochemistry
Food diary and instructions followed by consultation/evaluation with a certified dietician (Diary)
Feeding and Gastroenterologic Checklist (FGC)
Columbia Suicide Severity Rating Scale (C-SSRS)
Child Health Questionnaire-Parent Form 50 (CHQ)
Epworth Sleepiness Scales (ESS)
Study medications will be dispensed (MedDis)
Study medications will be collected
Adverse Events will be elicited and recorded (AE)
Concomitant medications will be elicited and recorded (ConMed)
Assessment of treatment effect (TE: intolerable, remediable, inadequate, adequate, optimal)

If the child reaches his or her optimal benefit or the maximum tolerated dose at a lower dose (i.e., at an earlier week), that dose will be continued to the end of the titration phase. The maintenance phase will begin at Week 4 at that dose.

The maintenance phase starts with the Week 4 assessments, which will include evaluation of interval behavioral, gastroenterologic, and medical changes; adverse events will be elicited, recorded, and managed; concomitant medications will be elicited and recorded; behavioral checklists will be completed; specific lab tests including peak and trough pK melatonin levels will be obtained; evaluation of gastrointestinal problems; evaluation of diet; assessment of any suicidal tendencies; measurement of daytime sleepiness.

The maintenance phase ends with the Week 7/Termination assessments, which will include evaluation of interval behavioral, gastroenterologic, and medical changes; adverse events will be elicited, recorded, and managed; concomitant medications will be elicited and recorded; behavioral checklists will be completed; specific lab tests including peak and trough pK melatonin levels will be obtained; evaluation of gastrointestinal problems; evaluation of diet; assessment of any suicidal tendencies; measurement of daytime sleepiness.

At the Week 7/Termination assessments, a schedule will be provided to the family for weaning of the study medication: For days 1-3 after the Week 7/Termination visit, the child will be administered 0.1 mg/kg (group 1) or placebo (group 2) morning, lunchtime, and at bedtime. For days 4-6 after the Week 7/Termination visit, the child will be administered 0.1 mg/kg or placebo in the morning and at bedtime. For days 7-9 after the Week 7/Termination visit, the child will be administered 0.1 mg/kg or placebo at bedtime. Day 10 after the Week 7/Termination visit will be the first day off of study medication. An alternative scenario, at the termination of dosing and unblinding of the dosage groups, patents may elect to continue treatment with study medication in an open label phase of the study. In the event that this option is selected, patients will continue to receive the previously administered dosage regimen (four daily doses).

At the Post-Termination visit (day 13±2 days after Week 7/Termination visit), the child will be evaluated for behavioral, medical, and gastroenterologic changes; adverse events will be elicited, recorded, and managed; concomitant medications will be elicited and recorded.

35 days±5 days after the last dose of study medication, a post-study phone call or a study visit will be conducted. At that time, the child will be evaluated for behavioral, medical, and gastroenterologic changes; adverse events will be elicited, recorded, and managed; concomitant medications will be elicited and recorded.

Statistics.

For individual treatment arms, point estimates of median and mean change from baseline to week 1, 2, 3, 4 and 7 will be calculated for the primary endpoint, along with associated confidence intervals. The planned sample size will thus be driven primarily by the desired precision of the parameter estimates for endpoints, and their associated variances.

Clinical Chemistry Assessments.

The systemic bioavailability of MEL and zinc will be evaluated based upon plasma and urine concentrations that will be measured by ELISA and AAS, respectively. The pharmacodynamics and efficacy of MEL and zinc will be determined experimentally according to changes in the production of malondialdehyde (MDA), a marker of lipid peroxidation, as evidence of their antioxidant activities (Kedziora-Kornatowska, K., Szewczyk-Golec, K., Czuczejko, J., van Marke de, L. K., Pawluk, H., Motyl, J., Karasek, M., and Kedziora, J. (2007). Effect of melatonin on the oxidative stress in erythrocytes of healthy young and elderly subjects. J. Pineal Res. 42, 153-158; Velkov, Z. A., Velkov, Y. Z., Galunska, B. T., Paskalev, D. N., and Tadjer, A. V. (2009). Melatonin: Quantum-chemical and biochemical investigation of antioxidant activity. Eur. J. Med. Chem. 44, 2834-2839). In addition, the effect of zinc supplementation on the activity of the zinc-dependent enzyme 5′-nucleotidase in plasma also will be assessed (Bales, C. W., DiSilvestro, R. A., Currie, K. L., Plaisted, C. S., Joung, H., Galanos, A. N., and Lin, P. H. (1994). Marginal zinc deficiency in older adults: responsiveness of zinc status indicators. J. Am. Coll. Nutr. 13, 455-462). Fecal calprotectin will be measured by ELISA using a commercially available test kit.

Intestinal permeability will be assessed by measuring urinary recovery of lactulose and mannatol, following oral administration. The ratio of lactulose to mannitol in urine is widely accepted as a measure of intestinal permeability (Camilleri, M., Nadeau, A., Lamsam, J., Nord, S. L., Ryks, M., Burton, D., Sweetser, S., Zinsmeister, A. R., and Singh, R. (2010). Understanding measurements of intestinal permeability in healthy humans with urine lactulose and mannitol excretion. Neurogastroenterol. Motil. 22, e15-e26; Benjamin, J., Makharia, G. K., Ahuja, V., Kalaivani, M., and Joshi, Y. K. (2008). Intestinal permeability and its association with the patient and disease characteristics in Crohn's disease. World J. Gastroenterol. 14, 1399-1405; Vilela, E. G., Tones, H. O., Ferrari, M. L., Lima, A. S., and Cunha, A. S. (2008). Gut permeability to lactulose and mannitol differs in treated Crohn's disease and celiac disease patients and healthy subjects. Braz. J. Med. Biol. Res. 41, 1105-1109) and is well-tolerated by pediatric patients (Hamilton, I., Hill, A., Bose, B., Bouchier, I. A., and Forsyth, J. S. (1987). Small intestinal permeability in pediatric clinical practice. J. Pediatr. Gastroenterol. Nutr. 6, 697-701). Briefly, subjects are given a single oral administration of 100 ml of a solution containing 5 g of lactulose and 2 g of mannitol, and urine is collected for 5 hours following ingestion. Concentrations of lactulose and mannitol in urine will be determined by high performance liquid chromatography as described previously (Vilela, E. G., Tones, H. 0., Ferrari, M. L., Lima, A. S., and Cunha, A. S. (2008). Gut permeability to lactulose and mannitol differs in treated Crohn's disease and celiac disease patients and healthy subjects. Braz. J. Med. Biol. Res. 41, 1105-1109). Studies indicate that as a group, healthy children with normal GI function typically display a mean urine lactulose:mannitolratio of less than 0.035.

Genomics Assessments.

The genomics assessments will evaluate the expression of specific methyltransferase genes that may be compromised in patients with ASD (Cai, G., Edelmann, L., Goldsmith, J. E., Cohen, N., Nakamine, A., Reichert, J. G., Hoffman, E. J., Zurawiecki, D. M., Silverman, J. M., Hollander, E., Soorya, L., Anagnostou, E., Betancur, C., and Buxbaum, J. D. (2008). Multiplex ligation-dependent probe amplification for genetic screening in autism spectrum disorders: efficient identification of known microduplications and identification of a novel microduplication in ASMT. BMC. Med. Genomics. 1:50., 50). These assessments will utilize specific single nucleotide polymorphisms (SNPs) that have been observed in the ASMT gene (Melke, J., Goubran, B. H., Chaste, P., Betancur, C., Nygren, G., Anckarsater, H., Rastam, M., Stahlberg, O., Gillberg, I. C., Delorme, R., Chabane, N., Mouren-Simeoni, M. C., Fauchereau, F., Durand, C. M., Chevalier, F., Drouot, X., Collet, C., Launay, J. M., Leboyer, M., Gillberg, C., and Bourgeron, T. (2008). Abnormal melatonin synthesis in autism spectrum disorders. Mol. Psychiatry. 13, 90-98; Jonsson, L., Ljunggren, E., Bremer, A., Pedersen, C., Landen, M., Thuresson, K., Giacobini, M., and Melke, J. (2010). Mutation screening of melatonin-related genes in patients with autism spectrum disorders. BMC. Med. Genomics. 3:10., 10).

The foregoing example(s) and description of the preferred embodiment should be taken as illustrating, rather than limiting, the present invention as defined by the claims. As will be readily appreciated, numerous variations and combinations of the features set forth above can be utilized without departing from the present invention as set forth in the claims. Such variations are not regarded as a departure from the spirit and scope of the invention, and all such variations are intended to be included within the scope of the following claims.

All references cited herein are incorporated by reference herein in their entireties.

1. A composition comprising melatonin and zinc.

2. The composition of claim 1, wherein the form of zinc is selected from the group consisting of zinc, zinc acetate, zinc gluconate, zinc citrate, zinc chloride and combinations thereof.

3. The composition of claim 1, wherein said melatonin and zinc are present in a molar ratio ranging from about 1:1 to about 1:1.2.

4. The composition of claim 1, wherein said melatonin and zinc are present in a molar ratio of about 1:1.

5. The composition of claim 4, wherein said melatonin and zinc are in a powdered form and mixed into a powdered mixture.

6. The composition of claim 5, wherein said powdered mixture is formulated with a sufficient amount of at least one excipient to produce particles.

7. The composition of claim 6, wherein said particles are coated with an enteric coating.

8. The composition of claim 7, wherein said enteric coating is selected from the group consisting of fatty acids, waxes, shellacs, plastics, plant fibers, polymers, and combinations thereof.

9. A composition comprising a melatonin-zinc complex.

10. The composition of claim 9, wherein said melatonin-zinc complex is formed by adding a soluble zinc salt to an alkaline solution of melatonin in the presence of excess alkali.

11. The composition of claim 1 or 9 wherein said composition further comprises probiotic materials.

12. The composition of claim 11, wherein said probiotic materials comprise at least one natural, semisynthetic, or transgenic strain of bacteria.

13. The composition of claim 11, wherein said probiotic materials comprise at least one natural, semisynthetic, or transgenic strain of yeast.

14. The composition of claim 11 wherein said probiotic materials comprise bacteria and yeast.

15. A dosage form comprising the composition of any one of claims 1-14.

16. The dosage form of claim 15 comprising particles of said composition.

17. The dosage form of claim 16, wherein said particles are coated with an enteric coating.

18. The dosage form of claim 17 wherein said coated particles are encapsulated.

19. A method of treating or preventing mercury toxicity comprising administering to a subject in need thereof a therapeutically effective amount of the composition of any one of claims 1-14.

20. A method of treating or preventing a condition selected from the group consisting of autism, autism spectrum disorders, Alzheimer's disease, and Parkinson's disease, comprising administering to a subject in need thereof a therapeutically effective amount of the composition of any one of claims 1-14.

21. A method of treating or preventing a condition selected from the group consisting of inflammation, mitochondrial dysfunction, and zinc deficiency comprising administering to a subject in need thereof a therapeutically effective amount of the composition of any one of claims 1-14.

22. A method of treating or preventing a condition associated with exposure to environmental heavy metals comprising administering to a subject in need thereof a therapeutically effective amount of the composition of any one of claims 1-14.

23. The method of treatment of any one of claims 20-22, wherein said composition is administered orally.

24. The method of treatment of any one of claims 20-22, wherein said subject is administered an amount of said composition providing from about 0.01 mg/kg to about 10 mg/kg melatonin one to four times per day.

25. The method of treatment of any one of claims 20-22, wherein said subject is administered an amount of said composition providing about 0.1 mg/kg of melatonin four times per day.

26. A method of making a dosage form comprising the steps of: combining melatonin and zinc to form a mixture, adding to said mixture an amount of at least one excipient sufficient to form particles, coating said particles with an enteric coating, and encapsulating said coated particles.
